(19) United States
(12) Patent Application Publication
(10) Pub. No.: US 2010/0281761 A1
(43) Pub. Date: Nov. 11, 2010

(54) ENERGY EFFICIENT AND GREENHOUSE GAS EFFICIENT BIOFUEL

(76) Inventor: Eric John Rey, Berkeley, CA (US)

Correspondence Address: Arcadia Biosciences, Inc., 2390 E. Camelback Rd., Suite 440, Phoenix, AZ, 85016, US

(21) Appl. No.: 12/679,441

(22) PCT Filed: Sep. 22, 2008
(86) PCT No.: PCT/US08/77268

§ 371 Date: Mar. 22, 2010

Related U.S. Application Data
(60) Provisional application No. 60/974,377, filed on Sep. 21, 2007.

(51) Int. Cl. C10L 1/182; C07C 27/06

(52) U.S. Cl. 44/386; 568/884

(57) ABSTRACT

An energy efficient biofuel a net energy value (NEV), which net energy value is calculated with a disclosed NEV formula and set of calculations. The energy efficient biofuel comprises ethanol that is derived from a genetically modified crop that is produced with a genetically modified seed. The Crop Yield per Nitrogen Application Rate of the genetically modified crop is at least 11% higher than the Crop Yield per Nitrogen Application Rate of a control crop. The irrigation, plant density, plant species, plant variety, and residual nitrogen of the genetically modified crop and the control crop are substantially the same.

### CROSS-REFERENCE TO RELATED APPLICATIONS

This application claims priority to PCT Application No. PCT/US2008/077268, filed Sep. 22, 2008, which claims priority to U.S. Provisional Application No. 60/974,377, filed Sep. 21, 2007, all of which are hereby incorporated by reference herein.

### FIELD OF THE INVENTION

The present application generally relates to agricultural based biofuels and, more particularly, to the calculation and optimization of the energy efficiency and greenhouse gas emissions efficiency of agricultural based biofuels.

### BACKGROUND

Energy security and climate change concerns require large-scale substitution of petroleum-based fuels. To be a viable alternative, a biofuel should provide a positive net energy value, have environmental benefits, be economically competitive, and be producible in large quantities without reducing food supplies. Today, ethanol and biodiesel are the two most widely used alternative fuels in the United States.

Ethanol is currently produced from corn grain in the United States and offers the opportunity to partially replace conventional gasoline in motor fuels. In 2005, the U.S. produced 3.9 billion gallons of ethanol (in 2006, 4.8 billion gallons), almost entirely based on the use of corn grain. Two federal policies are further motivating greater use of ethanol: a $0.51 tax credit per gallon of ethanol used as motor fuel and a mandate for up to 7.5 billion gallons of “renewable fuel” to be used in gasoline by 2012, the latter included in the Energy Policy Act (EPACT 2005).

Biodiesel is the fastest growing alternative fuel in the United States and worldwide. Worldwide production of biodiesel has seen an annualized growth of 33% over the last couple of years soaring from about 0.3 million tons in 1995 to nearly 7 million tons in 2006.

Thus, the energy and environmental implications of these biofuels are more important than ever.

Unfortunately, compared to gasoline these biofuels do not offer substantial improvements in overall environmental impact, as measured by net greenhouse gas (GHG) emissions and energy efficiency (net energy value). Studies reporting net energy values and net GHG emissions for corn grain ethanol vary, with some studies concluding that ethanol has higher net GHG emissions and lower net energy value than gasoline.

Therefore, optimization of the energy efficiency and greenhouse gas emissions efficiency of agricultural based biofuels is needed to reduce the environmental impact of agricultural based biofuels.

### SUMMARY

In one embodiment, a biofuel including ethanol is provided. The ethanol has a net energy value (NEV). The NEV of the ethanol is calculated using the formula NEV (MJ/LFuel)=[Biofuel Energy (MJ/Lfuel)+Coproduct Energy (MJ/Lfuel)]−[[[ΣiεFarmInputs(Embodied Energyi (MJ/kg)×Application Ratei (kg/ha))+ΣiεFarmInputs(Transport Energyi (MJ/kg)×Application Ratei (kg/ha))+[Nitrogen Embodied Energy (MJ/kgN)×Nitrogen Application Rate (kgN/ha)]+[Nitrogen Transport Energy (MJ/kgN)×Nitrogen Application Rate (kgN/ha)]+Farm Direct Energy (MJ/ha)+Farm Labor Energy (MJ/ha)+Farm Labor Transport Energy (MJ/ha)+Farm Machinery Energy (MJ/ha)+Inputs Packaging Energy (MJ/ha)]/[Crop Yield (kg/ha)×Production Yield (Lfuel/kg)]]+Biorefinery Energy (MJinput/Lfuel)]. In one embodiment, the FarmInputs includes one or more fertilizers, one or more herbicides, water, one or more insecticides, and seeds. In a further embodiment, the ethanol is derived from a genetically modified crop wherein the genetically modified crop is grown with a Crop Yield per Nitrogen Application Rate that is at least 8% higher than the Crop Yield per Nitrogen Application Rate of a non-genetically modified control crop. In this embodiment, the ethanol derived from the genetically modified crop exhibits at least an 11% increase in NEV compared to the NEV of ethanol produced from a non-genetically modified control crop. In a further embodiment, the increase in NEV of the ethanol produced from the genetically modified crop is a result of a change in the Nitrogen Application Rate of the genetically modified crop or a change in the Crop Yield of the genetically modified crop or a combination thereof In this embodiment, the Biofuel Energy, Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and the Biorefinery Energy of the NEV of the ethanol from the genetically modified crop and the NEV of the ethanol from the non-genetically modified control crop are substantially the same.

In a further embodiment, the genetically modified crop is transgenic. In a further embodiment, the genetically modified seed is chosen from wheat, Miscanthus, corn, rice, barley, sorghum, millets, oats, rye, and sugarcane, rapeseed, castor bean, sunflower, safflower, soybean, palm, sugar beets, and cotton.

In a further embodiment, the transgenic crop includes recombinant DNA wherein the recombinant DNA encodes one or more proteins involved in nitrogen metabolism. In a further embodiment, the one or more proteins are involved in nitrogen uptake or nitrogen assimilation. In yet a further embodiment, the one or more proteins includes alanine aminotransferase.

In a further embodiment, the ethanol has a net greenhouse gas emission. The net greenhouse gas emission of the ethanol is calculated using the formula net greenhouse gas emissions (kgCO2e/MJ)=[[[ΣᵢεFarmInputs(Input Emissionsᵢ (kgCO2e/kg)×Application Rateᵢ (kg/ha))+ΣᵢεFarmInputs(Transport Emissionsᵢ (kgCO2e/kg)×Application Rateᵢ (kg/ha))+[Fertilizer production emissions factor (kgCO2e/kgN)×Nitrogen application rate (kgN/ha)]+[Nitrification/denitrification emissions factor (kgCO2e/kgN)×Nitrogen Application Rate (kgN/ha)]+[Nitrogen Transport Emissions (kgCO2e/kgN)×Nitrogen Application Rate (kgN/ha)]+Farm Direct Emissions (kgCO2e/ha)+Farm Labor Transport Emissions (kgCO2e/ha)+Farm Machinery Emissions (kgCO2e/ha)+]/[Crop Yield (kg/ha)×Production Yield (L_{fuel}/kg)]]+Biorefinery Energy (kgCO2e/Lfuel)−Coproduct emissions (kgCO2e/L)]/HV of ethanol (MJ/L). In a further embodiment, FarmInputs includes one or more fertilizers, one or more herbicides, water, one or more insecticides, and seeds. In yet a further embodiment, the ethanol is derived from a genetically modified crop wherein the genetically modified crop is grown with a Crop Yield per Nitrogen Application Rate that is at least 8% higher than the Crop Yield per Nitrogen Application Rate of a non-genetically modified control crop. In a further embodiment, the ethanol derived from the genetically modified crop exhibits at least a 6% decrease in the net greenhouse gas emissions compared to the net greenhouse gas emissions of ethanol produced from the non-genetically modified control crop. In a further embodiment, the decrease in net greenhouse gas emissions of the ethanol produced from the genetically modified crop is a result of a change in the Nitrogen Application Rate of the genetically modified crop or a change in the Crop Yield of the genetically modified crop or a combination thereof. In a further embodiment, the Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and the Biorefinery Energy of the net greenhouse gas emissions of the ethanol from the genetically modified crop and the net greenhouse gas emissions of the ethanol from the non-genetically modified control crop are substantially the same.

In a further embodiment, a biofuel including biodiesel is provided. The biodiesel has a net energy value (NEV). In one embodiment, the NEV of the biodiesel is calculated using the formula NEV (MJ/LFuel)=[Biofuel Energy (MJ/Lfuel)+Coproduct Energy (MJ/Lfuel)]−[[[ΣiεFarmInputs(Embodied Energyi (MJ/kg)×Application Ratei (kg/ha))+ΣiεFarmInputs(Transport Energyi (MJ/kg)×Application Ratei (kg/ha))+[Nitrogen Embodied Energy (MJ/kgN)×Nitrogen Application Rate (kgN/ha)]+[Nitrogen Transport Energy (MJ/kgN)×Nitrogen Application Rate (kgN/ha)]+Farm Direct Energy (MJ/ha)+Farm Labor Energy (MJ/ha)+Farm Labor Transport Energy (MJ/ha)+Farm Machinery Energy (MJ/ha)+Inputs Packaging Energy (MJ/ha)]/[Crop Yield (kg/ha)×Production Yield (Lfuel/kg)]]+Biorefinery Energy (MJinput/Lfuel)]. In one embodiment, the FarmInputs include one or more fertilizers, one or more herbicides, water, one or more insecticides, and seeds. In a further embodiment, the biodiesel is derived from a genetically modified crop wherein the genetically modified crop is grown with a Crop Yield per Nitrogen Application Rate that is at least 8% higher than the Crop Yield per Nitrogen Application Rate of a non-genetically modified control crop. In a further embodiment, the biodiesel derived from the genetically modified crop exhibits at least a 10% increase in NEV compared to the NEV of biodiesel produced from the non-genetically modified control crop. In a further embodiment, the increase in NEV of the biodiesel produced from the genetically modified crop is a result of a change in the Nitrogen Application Rate of the genetically modified crop or a change in the Crop Yield of the genetically modified crop or a combination thereof. In a further embodiment, the Biofuel Energy, Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and the Biorefinery Energy of the NEV of the biodiesel from the genetically modified crop and the NEV of the biodiesel from the non-genetically modified control crop are substantially the same.

In a further embodiment, the biodiesel has a net greenhouse gas emission. The net greenhouse gas emission of the biodiesel is calculated using the formula net greenhouse gas emissions (kgCO2e/MJ)=[[[ΣᵢεFarmInputs(Input Emissionsᵢ (kgCO2e/k_{g})×Application Rateᵢ (kg/ha))+ΣᵢεFarmInputs(Transport Emissionsᵢ (kgCO2e/k_{g})×Application Rateᵢ (kg/ha))+[Fertilizer production emissions factor (kgCO2e/kgN)×Nitrogen application rate (kgN/ha)]+[Nitrification/denitrification emissions factor (kgCO2e/kgN)×Nitrogen Application Rate (kgN/ha)]+[Nitrogen Transport Emissions (kgCO2e/kgN)×Nitrogen Application Rate (kgN/ha)]+Farm Direct Emissions (kgCO2e/ha)+Farm Labor Transport Emissions (kgCO2e/ha)+Farm Machinery Emissions (kgCO2e/ha)+]/[Crop Yield (kg/ha)×Production Yield (L_{fuel}/kg)]]+Biorefinery Energy (kgCO2e/Lfuel)−Coproduct emissions (kgCO2e/L)]/HV of ethanol (MJ/L). In one embodiment, the FarmInputs include one or more fertilizers, one or more herbicides, water, one or more insecticides, and seeds. In a further embodiment, the biodiesel is derived from a genetically modified crop wherein the genetically modified crop is grown with a Crop Yield per Nitrogen Application Rate that is at least 8% higher than the Crop Yield per Nitrogen Application Rate of a non-genetically modified control crop, and wherein the biodiesel derived from the genetically modified crop exhibits at least a 10% decrease in the net greenhouse gas emissions compared to the net greenhouse gas emissions of biodiesel produced from the non-genetically modified control crop. In this embodiment, the decrease in net greenhouse gas emissions of the biodiesel produced from the genetically modified crop is a result of a change in the Nitrogen Application Rate of the genetically modified crop or a change in the Crop Yield of the genetically modified crop or a combination thereof In this embodiment, the Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and the Biorefinery Energy of the net greenhouse gas emissions of the biodiesel from the genetically modified crop and the net greenhouse gas emissions of the ethanol from the non-genetically modified control crop are substantially the same.

In a further embodiment, a method of producing biofuel is provided. The methods includes a) selecting genetically modified seed wherein the seed produces a genetically modified crop having a Crop Yield per Nitrogen Application Rate wherein the Crop Yield per Nitrogen Application Rate of the genetically modified crop is at least 8% higher than the Crop Yield per Nitrogen Application Rate of a non-genetically modified control crop; b) planting the genetically modified seed to produce the genetically modified crop; c) cultivating the genetically modified crop under conditions which maximize the Crop Yield per Nitrogen Application Rate of the genetically modified crop to produce a biofuel source; d) harvesting the biofuel source; e) preparing the biofuel source for processing to ethanol and f) processing the biofuel source to produce ethanol. The ethanol produced by the method has a net energy value (NEV). In one embodiment, the ethanol derived from the genetically modified crop exhibits at least an 11% increase in NEV compared to the NEV of ethanol produced from the non-genetically modified control crop. The increase in NEV of the ethanol produced from the genetically modified crop is a result of a change in the Nitrogen Application Rate of the genetically modified crop or a change in the Crop Yield of the genetically modified crop or a combination thereof In a further embodiment, the Biofuel Energy, Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and the Biorefinery Energy of the NEV of the ethanol from the genetically modified crop and the NEV of the ethanol from the non-genetically modified control crop are substantially the same. In the method, the net energy value is calculated with NEV (MJ/LFuel)=[Biofuel Energy (MJ/Lfuel)+Coproduct Energy (MJ/Lfuel)]−[[[ΣiεFarmInputs(Embodied Energyi (MJ/kg)×Application Ratei (kg/ha))+ΣiεFarmInputs(Transport Energyi (MJ/kg)×Application Ratei (kg/ha))+[Nitrogen Embodied Energy (MJ/kgN)×Nitrogen Application Rate (kgN/ha)]+[Nitrogen Transport Energy (MJ/kgN)×Nitrogen Application Rate (kgN/ha)]+Farm Direct Energy (MJ/ha)+Farm Labor Energy (MJ/ha)+Farm Labor Transport Energy (MJ/ha)+Farm Machinery Energy (MJ/ha)+Inputs Packaging Energy (MJ/ha)]/[Crop Yield (kg/ha)×Production Yield (Lfuel/kg)]]+Biorefinery Energy (MJinput/Lfuel)].

In a further embodiment, another method of producing biofuel is provided. The method includes a) selecting a genetically modified seed wherein the genetically modified seed produces a genetically modified crop having a Crop yield per Nitrogen Application Rate wherein the genetically modified crop is grown with a Crop Yield per Nitrogen Application Rate that is at least 8% higher than the Crop Yield per Nitrogen Application Rate of a non-genetically modified control crop; b) planting the genetically modified seed to produce the genetically modified crop; c) cultivating the genetically modified crop under conditions which maximize the Crop Yield per Nitrogen Application Rate of the genetically modified crop to produce a biofuel source; d) harvesting the biofuel source; e) preparing the biofuel source for processing to ethanol and f) processing the biofuel source to produce ethanol. The ethanol produced by the method has greenhouse gas emissions. The ethanol derived from the genetically modified crop exhibits at least a 6% decrease in the net greenhouse gas emissions compared to the net greenhouse gas emissions of ethanol produced from the non-genetically modified control crop. The decrease in net greenhouse gas emissions of the ethanol produced from the genetically modified crop is a result of a change in the Nitrogen Application Rate of the genetically modified crop or a change in the Crop Yield of the genetically modified crop or a combination thereof In a further embodiment, the Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and the Biorefinery Energy of the net greenhouse gas emissions of the ethanol from genetically modified crop and the net greenhouse gas emissions of the ethanol from the non-genetically modified control crop are substantially the same. In the method, the net greenhouse gas emissions (kgCO2e/MJ)=[[[ΣᵢεFarmInputs(Input Emissionsᵢ (kgCO2e/kg)×Application Rateᵢ (kg/ha))+ΣᵢεFarmInputs(Transport Emissionsᵢ (kgCO2e/kg)×Application Rateᵢ (kg/ha))+[Fertilizer production emissions factor (kgCO2e/kgN)×Nitrogen application rate (kgN/ha)]+[Nitrification/denitrification emissions factor (kgCO2e/kgN)×Nitrogen Application Rate (kgN/ha)]+[Nitrogen Transport Emissions (kgCO2e/kgN)×Nitrogen Application Rate (kgN/ha)]+Farm Direct Emissions (kgCO2e/ha)++Farm Labor Transport Emissions (kgCO2e/ha)+Farm Machinery Emissions (kgCO2e/ha)+]/[Crop Yield (kg/ha)×Production Yield (L_{fuel}/kg)]]+Biorefinery Energy (kgCO2e/Lfuel)−Coproduct emissions (kgCO2e/L)]/HV of ethanol (MJ/L), and the FarmInputs includes one or more fertilizers, one or more herbicides, water, one or more insecticides, and seeds.

In a further embodiment, a method of producing biofuel is provided. The method includes a) selecting genetically modified seed wherein the seed produces a genetically modified crop having a Crop Yield per Nitrogen Application Rate wherein the Crop Yield per Nitrogen Application Rate of the genetically modified crop is at least 8% higher than the Crop Yield per Nitrogen Application Rate of a non-genetically modified control crop; b) planting the genetically modified seed to produce the genetically modified crop; c) cultivating the genetically modified crop under conditions which maximize the Crop Yield per Nitrogen Application Rate of the genetically modified crop to produce a biofuel source; d) harvesting the biofuel source; e) preparing the biofuel source for processing to biodiesel and f) processing the biofuel source to produce biodiesel. The biodiesel has a net energy value (NEV). In the method, the biodiesel derived from the genetically modified crop exhibits at least a 10% increase in NEV compared to the NEV of biodiesel produced from the non-genetically modified control crop. The increase in NEV of the biodiesel produced from the genetically modified crop is a result of a change in the Nitrogen Application Rate of the genetically modified crop or a change in the Crop Yield of the genetically modified crop or a combination thereof The Biofuel Energy, Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and the Biorefinery Energy of the NEV of the biodiesel from genetically modified crop and the NEV of the biodiesel from the non-genetically modified control crop are substantially the same. The net energy value is calculated with NEV (MJ/LFuel)=[Biofuel Energy (MJ/Lfuel)+Coproduct Energy (MJ/Lfuel)]−[[[ΣiεFarmInputs(Embodied Energyi (MJ/kg)×Application Ratei (kg/ha))+ΣiεFarmInputs(Transport Energyi (MJ/kg)×Application Ratei (kg/ha))+[Nitrogen Embodied Energy (MJ/kgN)×Nitrogen Application Rate (kgN/ha)]+[Nitrogen Transport Energy (MJ/kgN)×Nitrogen Application Rate (kgN/ha)]+Farm Direct Energy (MJ/ha)+Farm Labor Energy (MJ/ha)+Farm Labor Transport Energy (MJ/ha)+Farm Machinery Energy (MJ/ha)+Inputs Packaging Energy (MJ/ha)]/[Crop Yield (kg/ha)×Production Yield (Lfuel/kg)]]+Biorefinery Energy (MJinput/Lfuel)]. The FarmInputs includes one or more fertilizers, one or more herbicides, water, one or more insecticides, and seeds.

In a further embodiment, another method of producing biofuel is provided. The method includes: a) selecting a genetically modified seed wherein the genetically modified seed produces a genetically modified crop having a Crop yield per Nitrogen Application Rate wherein the genetically modified crop is grown with a Crop Yield per Nitrogen Application Rate that is at least 8% higher than the Crop Yield per Nitrogen Application Rate of a non-genetically modified control crop, b) planting the genetically modified seed to produce the genetically modified crop; c) cultivating the genetically modified crop under conditions which maximize the Crop Yield per Nitrogen Application Rate of the genetically modified crop to produce a biofuel source; d) harvesting the biofuel source; e) preparing the biofuel source for processing to biodiesel and f) processing the biofuel source to produce biodiesel wherein the biodiesel has greenhouse gas emissions In one embodiment, the biodiesel derived from the genetically modified crop exhibits at least a 10% decrease in the net greenhouse gas emissions compared to the net greenhouse gas emissions of biodiesel produced from the non-genetically modified control crop, and wherein the decrease in net greenhouse gas emissions of the biodiesel produced from the genetically modified crop is a result of a change in the Nitrogen Application Rate of the genetically modified crop or a change in the Crop Yield of the genetically modified crop or a combination thereof and the Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and the Biorefinery Energy of the net greenhouse gas emissions of the biodiesel from the genetically modified crop and the non-genetically modified control crop are substantially the same. The Net greenhouse gas emissions are calculated using the formula: net greenhouse gas emissions (kgCO2e/MJ)=[[[ΣᵢεFarmInputs(Input Emissionsᵢ (kgCO2e/kg)×Application Rateᵢ (kg/ha))+ΣᵢεFarmInputs(Transport Emissionsᵢ (kgCO2e/kg)×Application Rateᵢ (kg/ha))+[Fertilizer production emissions factor (kgCO2e/kgN)×Nitrogen application rate (kgN/ha)]+[Nitrification/denitrification emissions factor (kgCO2e/kgN)×Nitrogen Application Rate (kgN/ha)]+[Nitrogen Transport Emissions (kgCO2e/kgN)×Nitrogen Application Rate (kgN/ha)]+Farm Direct Emissions (kgCO2e/ha)++Farm Labor Transport Emissions (kgCO2e/ha)+Farm Machinery Emissions (kgCO2e/ha) +]/[Crop Yield (kg/ha)×Production Yield (L_{fuel}/kg)]]+Biorefinery Energy (kgCO2e/Lfuel)−Coproduct emissions (kgCO2e/L)]/HV of ethanol (MJ/L). The FarmInputs includes one or more fertilizers, one or more herbicides, water, one or more insecticides, and seeds.

### BRIEF DESCRIPTION OF THE DRAWING

FIG. 1 depicts corn-based ethanol net GHG emissions in (gCO₂e/L-ethanol);

FIG. 2a depicts a relationship between nitrogen application and corn crop yield;

FIG. 2b depicts a relationship between nitrogen application and canola crop yield;

FIG. 3 depicts a relationship between percent reduction in nitrogen application and net GHG emissions (gCO₂e/MJ-ethanol);

FIG. 4 depicts a relationship between percent reduction in nitrogen application and net energy (MJ/L-ethanol);

FIG. 5 depicts one variation of a process of producing an energy efficient and/or GHG emissions efficient ethanol source; and

FIG. 6 depicts one variation of a plant growing system in which a genetically modified plant is grown at a higher yield per nitrogen input level.

### DETAILED DESCRIPTION OF PREFERRED EMBODIMENTS

In order to provide a more thorough understanding of the present invention, the following description sets forth numerous specific details, such as specific methods, parameters, examples, and the like. It should be recognized, however, that such description is not intended as a limitation on the scope of the present invention, but is intended to provide a better understanding of the exemplary variations. Calculations and analysis of New Energy Value and Greenhouse Gas Emissions are adopted from “Ethanol Can Contribute to Energy and Environmental Goals” by Alexander E. Farrell, Richard J. Plevin, Brian T. Turner, Andrew D. Jones, Michael O′Hare, and Daniel M. Kammen (2006) and the model entitled: ERG Biofuel Analysis Meta-Model (EBAMM), version 1.1, Energy and Resources Group and Richard & Rhoda Goldman School of Public Policy, University of California at Berkeley (2006), which is, incorporated by reference herein in its entirety. In particular, the values from the “Ethanol Today” case of EBAMM were utilized for the ethanol calculations and amended values from the “Ethanol Today” case of EBAMM as described in Table 3 were utilized for the biodiesel calculations.

a. Net Energy Value

Net Energy Value (NEV) is an accounting calculation that aims to find the sum total of energy necessary to produce a composition or sustain a process, including raw material extraction or creation, transport, and manufacturing or processing, and the sum total of the energy gained from such composition or process. In one exemplary method for calculating the NEV of a biofuel, or a component thereof such as ethanol, the energy inputs and energy outputs are measured and calculated as mega-joules per liter of biofuel as described in Eq. 1.

NEV(MJ/I_{fuel})=Output Energy(MJ_{fuel}/L_{Fuel})−Input Energy(MJ_{Input}/I_{Fuel}) (1)

The energy inputs for biofuel or a component thereof such as ethanol mainly include the energy utilized in the agricultural phase, where the biofuel source crop is planted, cultivated, and harvested, and the bio-refining phase, where the biofuel source is transported to a refinery and processed to form biofuel, as described in Eq. 2.

$$Input Energy ( {MJ}_{input} \text{/} {I}_{fuel} ) = \frac{Agricultural Energy ( {MJ}_{input} \text{/} ha )}{Net Yield ( {L}_{fuel} \text{/} ha )} + Biorefinery Energy ( {MJ}_{input} \text{/} {I}_{fuel} ) \qquad ( 2 )$$

Energy inputs for the agriculture phase are measured and calculated as mega-joules per hectare. Then energy inputs are divided by Net Yield, discussed below, which is a calculation of the portion of biofuel source crop utilized for biofuel production and yield in liters of biofuel from such portion.

Agricultural energy (in terms of mega-joules per hectare) can be calculated using Eq. 3 below.

$$Agricultural Energy ( MJ \text{/} ha ) = {\sum }_{i ∈ FarmInputs} ( Embodied {Energy}_{i} ( MJ \text{/} kg ) \times Application {Rat}_{i} e ( kg \text{/} ha ) ) + {\sum }_{i ∈ FarmInputs} ( Transport {Energy}_{i} ( MJ \text{/} kg ) \times Application {Rat}_{i} e ( kg \text{/} ha ) ) + Farm Direct Energy ( MJ \text{/} ha ) + Farm Labor Energy ( MJ \text{/} ha ) + Farm Labor Tran spo rt Energy ( MJ \text{/} ha ) + Farm Machinery Energy ( MJ \text{/} ha ) + Inputs Packaging Energy ( MJ \text{/} ha ) \qquad ( 3 )$$

The agricultural energy is calculated with two aggregate calculations of the net energy for each farm input and the energy to transport each input to the farm. Primary farm inputs include one or more fertilizers. The fertilizers may contain, one or more of nitrogen (e.g. as elemental N), phosphorus (as, e.g., P₂O₅), and potassium (as, e.g. K₂O); agricultural lime (crushed limestone, CaCO₃); herbicides; insecticides; and seeds. The net energy of each farm input is calculated with the embodied energy for each input (in terms of mega-joules per kilogram) and the application rate of each input (in terms of kilograms per hectare). The transportation energy is calculated with the transport energy for each input (in terms of mega-joules per kilogram) and the application rate of each input (in terms of kilograms per hectare). Direct energy can include gasoline, diesel, liquefied petroleum gas (LPG), and electricity. Labor energy, labor transport, farm machinery, and inputs packaging can also be estimated and included in the agricultural energy calculation. Labor energy is calculated with the amount of human labor (in terms of mega-joules per hour) and the application of human labor to the farm (in terms of hours per hectare). Farm labor transport energy is calculated with the amount of energy to transport farm laborers roundtrip to the crop site. Farm machinery energy is calculated with the energy associated with the manufacture of equipment for crop production plus the energy in maintenance (in terms of mega-Joules) prorated per hectare. Inputs packaging energy is the amount on energy required to prepare farm inputs for use on the farm. Preparation of farm inputs includes the granulation and preparation of mixtures of nitrogen, phosphate and potash and can include the packaging of agricultural lime, herbicides, insecticides and seeds.

Net Yield can be calculated with Crop Yield (in terms of kilograms per hectare) and Conversion Yield (L/kg), as described in Eq. 4. Crop Yield is the portion of the plant harvested for ethanol production. Conversion Yield is the amount of ethanol produced at the biorefinery for a unit of corn input.

Net Yield(L/ha)=Crop Yield (kg/ha)×Ethanol Production Yield(L/kg) (4)

The energy consumed in producing biofuel from a biofuel source can be calculated with Eq. 5. Biorefinery energy is calculated in terms of mega-joules per liter of biofuel.

$$Biorefinery Energy ( {MJ}_{input} \text{/} {L}_{fuel} ) = Feedstock Transport Energy ( MJ \text{/} L ) + Electricity Inputs ( MJ \text{/} L ) + Coal Inputs ( MJ \text{/} L ) + Natural Gas Inputs ( MJ \text{/} L ) + Diesel Inputs ( MJ \text{/} L ) + Biomass Energy Inputs ( MJ \text{/} L ) + Process Water Inputs ( MJ \text{/} L ) + Effluent Water Energy ( MJ \text{/} L ) + Embodied Energy ( MJ \text{/} L ) \qquad ( 5 )$$

Output energy can be determined as the sum of the energy gained from the biofuel and any energy gained from a co-product or byproduct of the agricultural or refining phases.

$$Output Energy ( MJ \text{/} {I}_{fuel} ) = Fuel Energy ( MJ \text{/} {I}_{fuel} ) + Coproduct Energy ( MJ \text{/} {I}_{fuel} ) \qquad ( 6 )$$

Biofuel production yields various co-products, depending on the biofuel source and processes employed. For example, ethanol production from corn co-produces corn oil, distiller's dried grains with solubles, corn gluten feed, and/or corn gluten meal, depending on whether dry- or wet-milling is utilized. In another variation, biodiesel production from canola co-produces canola meal feed and glycerol. When these co-products have a positive economic value, they will displace competing products that also require energy to produce. For example, co-products produced from both current and anticipated increases in corn ethanol production are expected to be valuable feed products that will displace whole corn and soybean meal in animal feed.

Studies have significantly varied in determining a co-product energy credit for corn based ethanol production, ranging from 0 mega-joules per liter to −7.3 mega-joules per liter. The leading authority on co-product credits has adopted a value of −4.1 MJ/L: “Ethanol Can Contribute to Energy and Environmental Goals” by Alexander E. Farrell, Richard J. Plevin, Brian T. Turner, Andrew D. Jones, Michael O'Hare, and Daniel M. Kammen (2006) and the model entitled: ERG Biofuel Analysis Meta-Model (EBAMM), version 1.1, Energy and Resources Group and Richard & Rhoda Goldman School of Public Policy, University of California at Berkeley (2006), which are, in part, adopted and incorporated herein. In particular, the values from the “Ethanol Today” case of EBAMM were utilized for the ethanol calculations.

For canola and soybean based biodiesel, the assumptions and calculations of Hill,

J., E. Nelson, D. Tilman, S. Polasky and D. Tiffany, 2006, Environmental, economic, and energetic costs and benefits of biodiesel and ethanol biofuels, Proceedings of the National Academy of Sciences 103: 11206-210 (“Hill et al.”) (In particular, amended values from the “Ethanol Today” case as described in Table 3 were utilized for the biodiesel calculations.); and Jaeger, William K., Cross, Robin, & Egelkraut, Thorsten M., 2007, Biofuel Potential in Oregon: Background and Evaluation of Options, Department of Agricultural and Resource Economics Oregon State University (“Jaeger et al.”), are, adopted and incorporated by reference herein in their entirety.

b. Net Greenhouse Gas (GHG) Emissions

Net Greenhouse Gas (GHG) emissions can be calculated as the sum of the agricultural production emissions of the biofuel source, the biofuel production emissions, and the fuel emissions, minus co-product emissions as described in Eq. 7. The primary GHG emissions that are significant in biofuel agricultural and production phases are carbon dioxide (CO₂), methane (CH₄), and nitrous oxide (N₂O). GHG emissions can be aggregated on a carbon dioxide-equivalent (CO₂e) basis with values of 1 for CO₂, 23 for CH₄, and 296 for N₂O, according to the Intergovernmental Panel on Climate Change, Climate Change 2001: The Scientific Basis (Cambridge, UK: Cambridge University Press, 2001), which is hereby incorporated, in part, by reference.

$$G H G emissions ( {kgCO}_{2} e \text{/} MJ ) = [ \begin{matrix}\frac{Agricultural emissions ( {kgCO}_{2} e \text{/} ha )}{Net Yield ( {L}_{fuel} \text{/} ha )} + \\ Biorefinery emissions ( {kgCO}_{2} e \text{/} L ) - \\ Coproduct emissions ( {kgCO}_{2} e \text{/} L )\end{matrix} ] / H V of ethanol ( MJ \text{/} L ) \qquad ( 7 )$$

Primary agricultural GHG emissions arise from fertilizers containing, for example, nitrogen (as elemental N), phosphorus (as P₂O₅), and/or potassium (as K₂O). However, nitrogen fertilizers account for the overwhelming share of GHG emissions in the agricultural stage. Thus, agricultural emissions can be calculated with Eq. 8 with special attention to GHG emissions resulting from nitrogen fertilizer, as discussed below.

$$Agricultural Emissions ( {kgCO}_{2} e \text{/} Kg ) = [ \begin{matrix}\begin{matrix}\begin{matrix}\begin{matrix}\begin{matrix}{\sum }_{i ∈ FarmInputs} ( \begin{matrix}Input {Emissions}_{i} ( {kgCO}_{2} e \text{/} kg ) \times \\ Application {Rat}_{i} e ( kg \text{/} ha )\end{matrix} ) + \\ {\sum }_{i ∈ FarmInputs} ( \begin{matrix}Transport {Emissions}_{i} ( {kgCO}_{2} e \text{/} kg ) \times \\ Application {Rat}_{i} e ( kg \text{/} ha )\end{matrix} ) +\end{matrix} \\ Farm Direct Emissions ( {kgCO}_{2} e \text{/} ha ) +\end{matrix} \\ Farm Labor Emissions ( {kgCO}_{2} e \text{/} ha ) +\end{matrix} \\ Farm Labor Transport Emissions ( {kgCO}_{2} e \text{/} ha ) +\end{matrix} \\ Farm Machinery Emissions ( {kgCO}_{2} e \text{/} ha ) + \\ Inputs Packaging Emissions ( {kgCO}_{2} e \text{/} ha )\end{matrix} ] / [ CropYield ( kg \text{/} ha ) ] \qquad ( 8 )$$

Use of nitrogen fertilizer results in GHG emissions in two stages: fertilizer manufacture (primarily CO₂ emissions from energy use) and fertilizer application (primarily from N₂O emissions from nitrification and denitrification processes in soil). GHG emissions resulting from nitrogen fertilizer can be calculated on a per hectare basis as shown in Eqs. 9-11.

$$\begin{matrix}Nitrogen fertilizer emissions ( {kgCO}_{2} e \text{/} ha ) = Fertilizer production emissions ( {kgCO}_{2} e \text{/} ha ) + Soil related emissions ( {kgCO}_{2} e \text{/} ha ) + Fertilizer transport emissions ( {kgCO}_{2} e \text{/} ha ) + Fertilizer transport emissions ( {kgCO}_{2} e \text{/} ha ) & ( 9 ) \\ Fertilizer production emissions ( {kgCO}_{2} e \text{/} ha ) = Fertilizer production emissions factor ( {kgCO}_{2} e \text{/} kgN ) \times \\ Nitrogen application rate ( kgN \text{/} ha ) & ( 10 ) \\ Soil related emission ( {kgCO}_{2} e \text{/} haN ) = Nitrification \text{/} denitrification emissions factor ( {kgCO}_{2} e \text{/} kgN ) \times \\ Nitrogen application rate ( kgN \text{/} ha ) & ( 11 )\end{matrix}$$

GHG emissions from processing biofuel from a biofuel source can be calculated in accordance with Eq. 12. Biorefinery GHG emissions are calculated in terms of kilograms of CO₂ equivalent emissions per liter of biofuel.

$$Biorefinery Emissions ( {kgCO}_{2} e \text{/} L ) = Feedstock Transport ( {kgCO}_{2} e \text{/} L ) + Electricity Inputs ( {kgCO}_{2} e \text{/} L ) + Coal Inuts ( {kgCO}_{2} e \text{/} L ) + Natural Gas Inputs ( {kgCO}_{2} e \text{/} L ) + Diesel Inputs ( {kgCO}_{2} e \text{/} L ) + Biomass Inputs ( {kgCO}_{2} e \text{/} L ) + Process Water Inputs ( {kgCO}_{2} e \text{/} L ) + Effluent Water ( {kgCO}_{2} e \text{/} L ) + Capital ( plant and equipment ) ( {kgCO}_{2} e \text{/} L ) \qquad ( 12 )$$

c. Environmental Impact

While biofuels do help to relieve reliance on foreign petroleum, they do not currently offer substantial improvements in overall environmental impact compared to gasoline. For example, net greenhouse gas emissions and net energy values of corn-based ethanol are not significantly better than that of gasoline. In fact some studies suggest that the net GHG emissions of ethanol are higher than gasoline and that the net energy of ethanol is lower than gasoline. Gasoline has net GHG emissions of 94 g CO₂e per MJ and a net energy value of −0.24 MJ/L. Various studies have reported corn-based ethanol net greenhouse gas emissions between 62 and 120 g CO₂e per MJ (EBAMM's “Ethanol Today” value of 77 g CO₂e per MJ) and net energy values between 8.9 and -6.1 MEL (EBAMM's “Ethanol Today” value of 4.62 MJ/L). Biodiesel is reported to be more energy efficient and less of a polluter with a NEV of 122978 BTU/gallon (34.26 MJ/L) and 51698 gCO₂e/million BTU (49 gCO₂e/MJ) according to Hill et al. and Jaeger et al.; however, biodiesel still results in significant environmental impact.

While studies report varying net energy values and net GHG emissions for biofuels, it is clear that methods and improvements are necessary to raise their net energy value and lower the net GHG emissions.

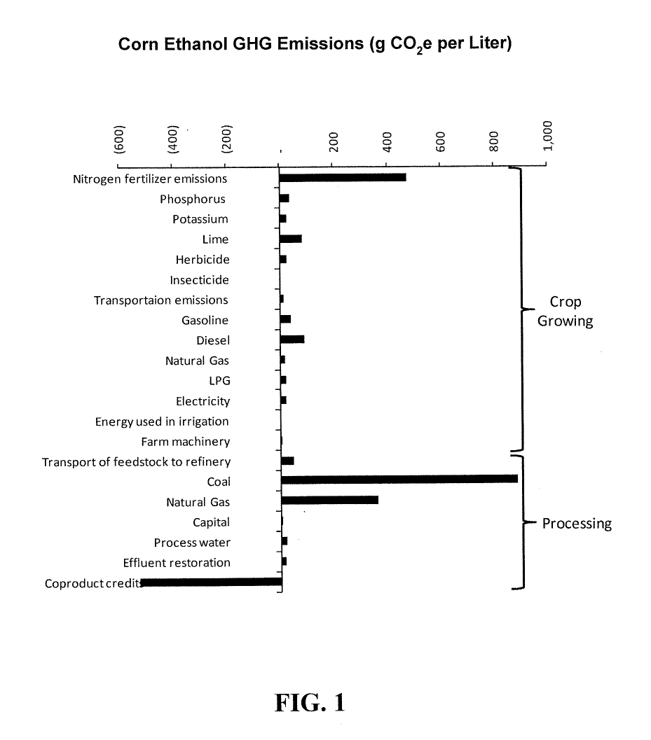

Approximately 30% of total GHG emissions from corn ethanol are estimated to arise from the use of nitrogen fertilizer in producing corn grain. Agricultural GHG emissions from nitrogen fertilizer are often in the form of nitrous oxide, a gas that has a Global Warming Potential that is 296 times greater than carbon dioxide (CO₂). Referring again to FIG. 1, it is clear that GHG emissions related to nitrogen fertilizer dominate all other agricultural emissions.

Lowering the nitrogen application rate per yield of a biofuel source crop will raise the net energy values and lower the net GHG emissions for the resulting biofuel. However, yield can be highly dependent on the ability of the plant to assimilate and utilize N during vegetative growth and then remobilize acquired N to the seed during the grain filling period. Thus, lowering the nitrogen application rate of a non-Nitrogen Use Efficient (NUE), non-genetically modified crop will eventually begin to lower the crop yield, possibly offsetting the environmental gains.

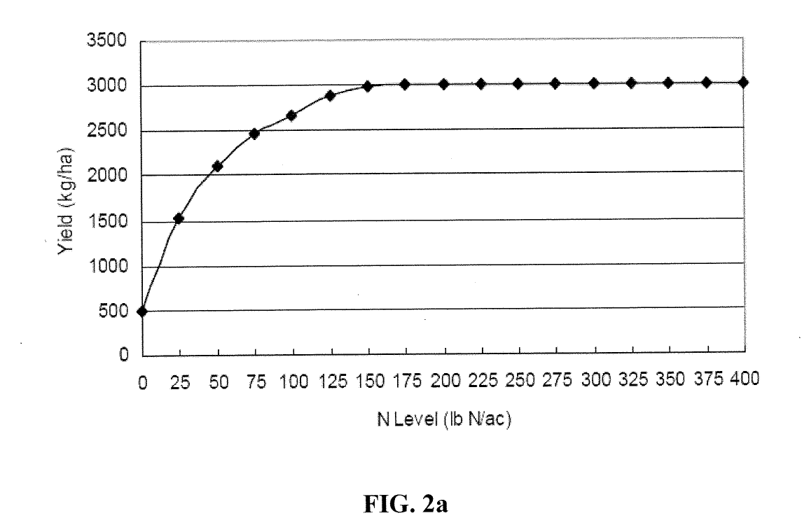

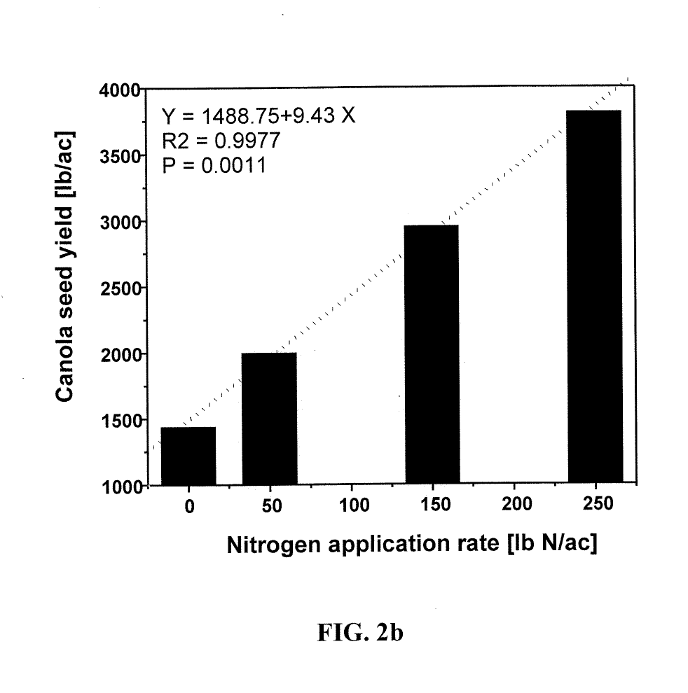

With reference to FIG. 2a, which is a graph of a nitrogen application versus corn crop yield developed by Jianqiang He at the Department of Agricultural and Biological Engineering of the University of Florida, crop yield is reported in kilograms per hectare and nitrogen application is reported in pounds of nitrogen per acre. In this example, crop yield was lower in fields where nitrogen application was below 150 lb N/ac and exponentially lower in fields where the nitrogen application was below 150 lb N/ac.

With reference to FIG. 2b, which is a graph of a nitrogen application versus canola crop yield, where crop yield is reported in pounds per acre and nitrogen application is reported in pounds of nitrogen per acre. In this example, as in the example above, lower nitrogen application rates lead to lower crop yields.

Lower yields can eliminate or even reverse increases in net energy value and decreases in net GHG emissions gained from lower application rates of nitrogen. For example, referring to Eq. 1, as nitrogen fertilizer application is reduced, the value of Input Energy is lowered. However, if the nitrogen application rate reduction lowers crop yield, then the amount of ethanol produced will also decrease, which will result in a decreased Output Energy. Thus, to enjoy significant increases in net energy value and decreases in net GHG emissions, the crop yield per nitrogen application rate needs to increase, if the irrigation, plant density, plant species, plant variety, and residual nitrogen of the crop and remains substantially the same.

Other factors that are important to the evaluation of crop yield are water irrigation, residual nitrogen, and plant density. Residual nitrogen is nitrogen that exists in the soil before additional nitrogen fertilizer is applied. Residual nitrogen concentrations vary with cropping and fertilizer history, and are continually changing in response to climatic conditions, soil organic matter content, farming, biological, and physical processes. Residual nitrogen in soil-water can contribute significant amounts of available nitrogen to the plant. Thus, soil that has a higher residual nitrogen level, in plant-available formations, may require less nitrogen application to achieve a similar crop yield.

Irrigation water can also contain nitrogen, mostly in the inorganic nitrate form. In general, increase in soil moisture enhances corn yield response to N fertilization, especially when high N rates are applied. In addition, N uptake is strongly influenced by water supply.

Both nitrogen in irrigation water and residual nitrogen in the soil can be determined by laboratory tests.

Plant density usually increases corn yield until an optimum number of plants per unit area is reached. Optimum plant density is related to variety, soil texture, soil moisture availability, N fertility, and other environmental factors. For example, some studies suggest that optimum plant density for corn production increases with the increase of available N up to a plant density of approximately 50000 plants per hectare.

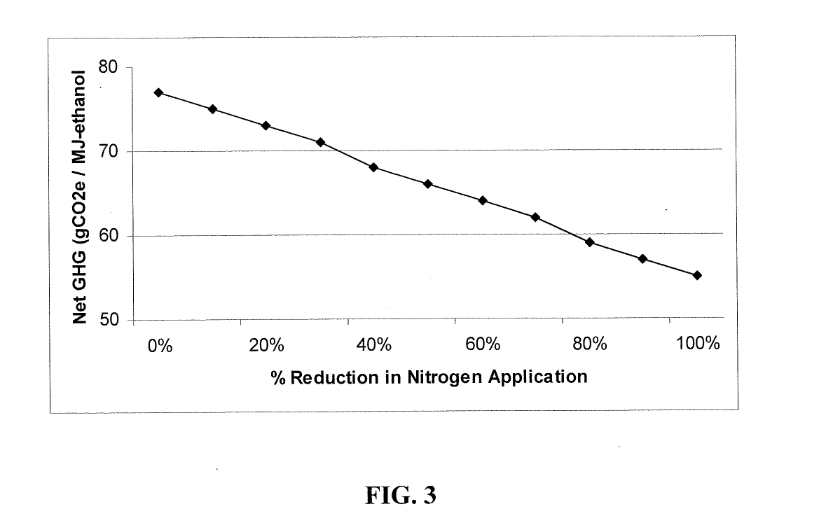

FIG. 3, which depicts a graph of the percent reduction in nitrogen application and net GHG emissions (gCO₂e/MJ-ethanol), shows the potential reduction of GHG emissions with decreased nitrogen application. This graph is derived using the foregoing calculations and the assumptions embodied in EBAMM's “Ethanol Today” case study. In particular, the nitrogen application rates of Eqs. 10 and 11 were varied. Referring to FIG. 3, a two-thirds reduction in N application results in EBAMM's “Ethanol Today” net GHG emissions value dropping from 77 g CO₂e per MJ to 62 g CO₂e per MJ for a 22% decrease.

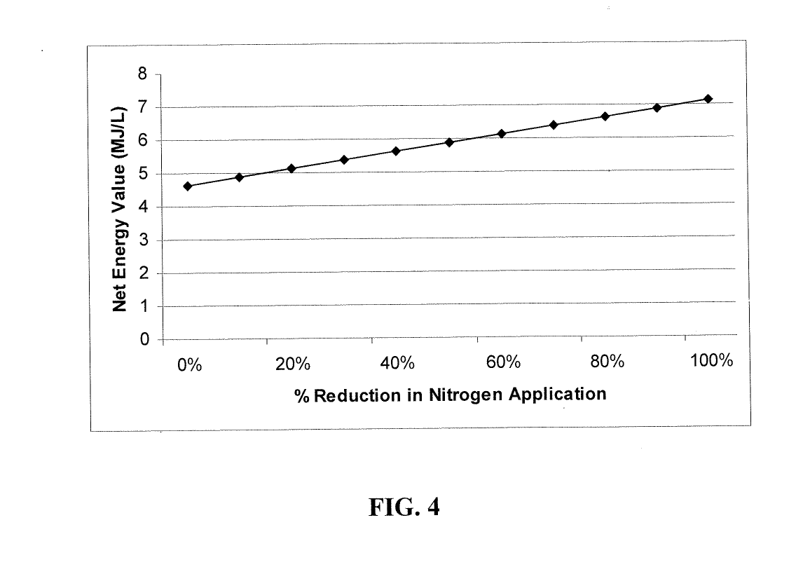

FIG. 4, which depicts a graph of the percent reduction in nitrogen application and net energy value (MEL-ethanol), shows the potential increase of net energy value with decreased nitrogen application. This graph is derived with the foregoing calculations and the assumptions embodied in EBAMM's “Ethanol Today” case study. In particular, the nitrogen application rate of Eq. 3 was varied. Referring to FIG. 4, a two-thirds reduction in N application results in EBAMM's “Ethanol Today” net energy value rising from 4.62 MEL to 6.29 MEL for a 31% increase.

Various farming techniques can be adopted to lower nitrogen application rates while maintaining crop yield. For example, in one variation, a “no till” seeding method can be utilized. A “no till” method simply removes the step of tilling the stubble from a previous crop prior to planting the next successive crop. Instead, the next crop is planted directly in the stubble. The stubble is very effective in holding beneficial fertilizer in place rather than allowing it to run off with excess water. This fertilizer-holding characteristic also serves to hold moisture within the soil regardless of terrain conditions.

Another method of lowing nitrogen application rates while maintaining crop yield is to utilize crops that require less nitrogen. Several approaches have been used to increase nitrogen use efficiency (NUE) in crop plants, including, traditional breeding and marker-assisted selection, and the use of gene constructs designed to improve specific aspects of NUE.

There are two major components of NUE, nitrogen (N) uptake efficiency and N utilization efficiency. N uptake efficiency is the percentage of fertilizer-applied N found in the plant at maturity and describes the ability of the source plant to mine and assimilate N from the soil. Alternatively, N utilization efficiency is the ratio of grain yield to plant N and indicates sink capacity for using acquired N.

Conventional plant breeding methods typically include the generation of a population of plants having desirable traits, evaluation and selection of superior individuals, and recombination of the superior individuals to generate a new population for subsequent cycles of selection and improvement. Marker-assisted selection and DNA fingerprinting techniques can potentially increase the efficiency of traditional breeding programs by speeding up the time of varietal release, lowering plant population requirements, and eliminating costly field evaluation. DNA fingerprinting tools such as RFLP, AFLP and microsatellites can be utilized together with information on genetic linkage to quantitative traits and the development of genetic maps. TILLING (Targeted Induced Local Lesions In Genomes) can also be utilized to efficiently identify useful mutations. The steps typically followed in TILLING are: (a) EMS mutagenesis; (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art.

Another approach to increase nitrogen in crop plants uses novel gene constructs to create transgenic crops designed to improve specific aspects of NUE. Such gene constructs include genes involved in nitrogen uptake and metabolism. As but one example, recombinant DNA encoding alanine aminotransferase may be introduced into plants as described in US Patent application 20070162995 which is hereby incorporated by reference in its entirety. Methodologies introducing recombinant nucleic acids into plants are generally known in the art and have been previously described. For example, recombinant DNA can be introduced into plants, using tumor-inducing (Ti) plasmid vectors. Other methods utilized for recombinant DNA delivery involve the use of PEG mediated protoplast transformation, electroporation, microinjection whiskers, and biolistics or microprojectile bombardment for direct DNA uptake. Biolistics (otherwise known as Particle Bombardment) involves directly “shooting” a piece of DNA into the recipient plant tissue. This is carried out using a gene gun. Tungsten or gold beads (which are smaller than the plant cells themselves) are coated in the gene of interest and fired through a stopping screen, accelerated by helium, into the plant tissue. The particles pass through the plant cells, leaving the DNA inside.

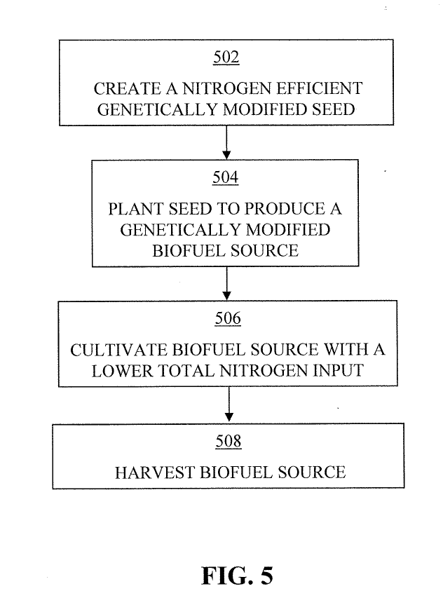

FIG. 5 is one variation of a process of producing energy efficient and/or GHG emissions efficient biofuel source. In step 502, a genetically modified seed is created that is capable of producing a higher yield per nitrogen input level.

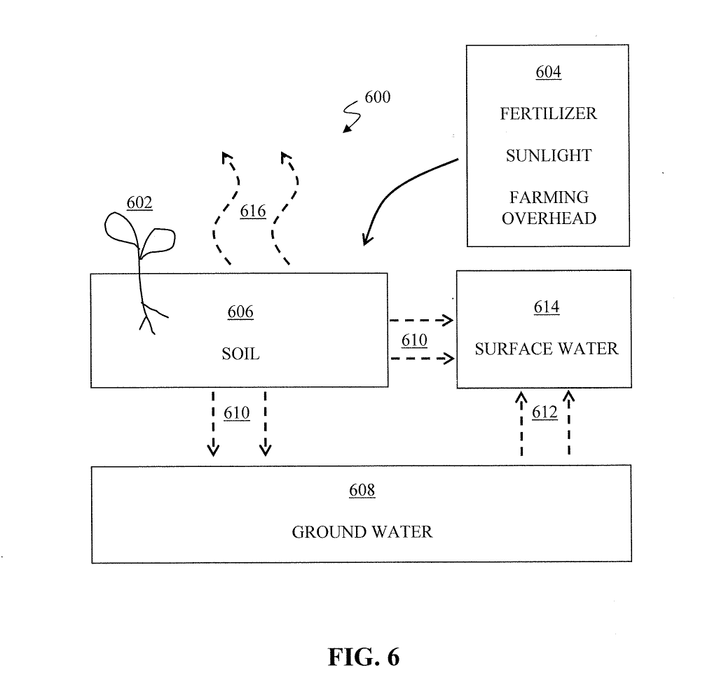

With reference to FIG. 6, system 600 is one variation of a plant growing system.

System 600 is depicted with plant 602, which could represent any genetically modified plant or crop. One example of plant 602 could be, but is not limited to, a variety of wheat, corn, rice, barley, sorghum, millets, oats, rye, sugarcane, Miscanthus, rapeseed, castor bean, sunflower, safflower, soybean, palm, sugar beets, cotton, and the like, that has been genetically modified to enhance nitrogen utilization, effectively reducing the amount of nitrogen required for crop development (or effectively raising the yield with the same amount of nitrogen). The genetic modification could be a traditional breeding and selection method, various transgenic methods, or any other method of manipulating genetics, as discussed above.

With reference again to FIG. 5, step 504 entails planting the created genetically modified seed to produce a genetically modified biofuel source crop. Step 506 entails cultivating the genetically modified biofuel source crop under conditions which produce increase the yield per total nitrogen input level than a comparable, non-genetically modified biofuel source crop.

As discussed above, GHG emissions can be significantly reduced and net energy can be significantly increased by utilizing genetically modified crops that are more nitrogen efficient. As depicted in FIG. 6, several energy inputs 604 are utilized for crop development. These inputs include, but are not limited to, sunlight, water, nitrogen fertilizer, and farming overhead. Only a fraction of the nitrogen that is introduced into soil 606 may be utilized by plant 602. Some of the nitrogen input 604 escapes in water 610 or is volatilized and enters the atmosphere as a gas 616.

With reference again to FIG. 5, step 508 entails harvesting the energy efficient and/or GHG emissions efficient biofuel source. Harvesting methods will depend on the biofuel source crop. In one variation of the biofuel source, corn grain and corn stover can be utilized. In this case a producer may choose to harvest stover after grain, allowing it to dry in the fields or could use single-pass harvest and harvest the stover wet.

Corn based ethanol can be produced with the following steps: hydrolysis (optional), fermentation, distillation, and dehydration.

During hydrolysis carbohydrates such as cellulose and starch are converted into sugars. In some variations, hydrolysis methods include physical, chemical, biological treatment, or any combination of the foregoing. Non-limiting examples of physical treatment can include various types of milling, crushing, irradiation, steaming/steam explosion, and hydrothermolysis. Non-limiting examples of chemical treatment can include dilute acid, alkaline, organic solvent, ammonia, sulfur dioxide, carbon dioxide, and pH-controlled hydrothermolysis. Non-limiting examples of biological treatment can involve applying lignin-solubilizing microorganisms.

Ethanol can be produced by fermentation of the carbohydrates with yeast or bacteria like Escherichia coli or Clostridia. The fermentation of carbohydrates to acetone, butanol, and ethanol (ABE) by solventogenic microorganisms is well known in the art.

After fermentation the majority of water is removed from the solvent mixture by way of distillation. Subsequent to distillation dehydration can be used to remove more water. The dehydration processes can include: azeotropic distillation (consists of adding benzene or cyclohexane to the mixture), extractive distillation (consists of adding a ternary component which will increase ethanol relative volatility), or with the use of molecular sieves (consists of passing the ethanol vapor under pressure passes through a bed of molecular sieve beads).

The most widely used refining method for biodiesel is transesterification or alcoholysis of a vegetable oil. Other refining methods can include: supercritical processes, ultrasonic methods, and microwave methods. A transesterification or alcoholysis refining process includes catalytically reacting oils with a short-chain aliphatic alcohol, like methanol or ethanol.

In some variations, biodiesel can be produced, via transesterification refining, with the following steps: purification, neutralization, and transesterification. In the purification step water and all non-oils are removed. Non-oil material can be removed with a filer and water can be removed with a drying agent such as magnesium sulfate. In the neutralization step, free fatty acids are neutralized by: determining the concentration of free fatty acids present in the oil, calculating the quantity of base required to neutralize the free fatty acids, and adding the base (usually sodium hydroxide) to the oil. In the transesterification step, the alcohol (methanol or ethanol) is added to the solution of the oil, and the combined solution is heated.

Table 1 depicts a table of the percentage increase in net energy value (NEV) and percentage decrease in net greenhouse gas emissions for each percentage increase in crop yield and percentage decrease in the nitrogen application rate as compared to a control crop when calculated using the ERG Biofuel Analysis Meta-Model (EBAMM), version 1.1. In particular, the values from the “Ethanol Today” case of EBAMM are utilized. For example, an increase in crop yield of 10% with no change in nitrogen application will result in an 11% increase in NEV and a 4% decrease in net greenhouse gas emissions. In another example, an increase in crop yield of 10% with a decrease in nitrogen application of 20% will result in a 21% increase in NEV and a 10% decrease in net greenhouse gas emissions. The table demonstrates that NEV and greenhouse gas emissions can be controlled by changes in the nitrogen application rate, changes in the crop yield, or combinations thereof.

Table 2 depicts a table of the percentage increase in net energy value (NEV) and percentage decrease in net greenhouse gas emissions for each percentage increase in crop yield and percentage decrease in the nitrogen application rate as compared to a control crop for biodiesel when calculated using the ERG Biofuel Analysis Meta-Model (EBAMM), version 1.1. In particular, the amended values (shown in Table 3) from the “Ethanol Today” case of EBAMM are utilized. The table demonstrates that NEV and greenhouse gas emissions can be controlled by changes in the nitrogen application rate, changes in the crop yield, or combinations thereof.

Table 3 depicts a table of the changes made to the ERG Biofuel Analysis Meta-Model (EBAMM), version 1.1, “Ethanol Today” case of EBAMM for the biodiesel calculations.

TABLE 1
% Increase in Crop Yield

| | | 0% | | 5% | | 10% | | 15% | |
|---|---|---|---|---|---|---|---|---|---|
| | | % | % | % | % | % | % | % | % |
| | | Increase | Decrease | Increase | Decrease | Increase | Decrease | Increase | Decrease |
| | | in NEV | in GHG | in NEV | in GHG | in NEV | in GHG | in NEV | in GHG |
| % | 0% | 0% | 0% | 6% | 2% | 11% | 4% | 15% | 6% |
| Decrease | 10% | 5% | 3% | 11% | 5% | 16% | 7% | 20% | 9% |
| in Nitrogen | 20% | 11% | 6% | 16% | 8% | 21% | 10% | 25% | 11% |
| Application | 30% | 16% | 9% | 21% | 11% | 25% | 12% | 29% | 14% |
| Rate | 40% | 22% | 12% | 26% | 13% | 30% | 15% | 34% | 16% |
| | 50% | 27% | 15% | 31% | 16% | 35% | 18% | 39% | 19% |
| | 60% | 32% | 17% | 36% | 19% | 40% | 20% | 43% | 21% |
| | 70% | 38% | 20% | 41% | 22% | 45% | 23% | 48% | 24% |

| | | 20% | | 25% | | 30% | |
|---|---|---|---|---|---|---|---|
| | | % | % | % | % | % | % |
| | | Increase | Decrease | Increase | Decrease | Increase | Decrease |
| | | in NEV | in GHG | in NEV | in GHG | in NEV | in GHG |
| % | 0% | 20% | 8% | 24% | 10% | 27% | 11% |
| Decrease | 10% | 24% | 10% | 28% | 12% | 31% | 13% |
| in Nitrogen | 20% | 29% | 13% | 32% | 14% | 36% | 15% |
| Application | 30% | 33% | 15% | 37% | 16% | 40% | 18% |
| Rate | 40% | 38% | 18% | 41% | 19% | 44% | 20% |
| | 50% | 42% | 20% | 45% | 21% | 48% | 22% |
| | 60% | 47% | 22% | 49% | 23% | 52% | 24% |
| | 70% | 51% | 25% | 54% | 26% | 56% | 27% |

TABLE 2
% Increase in Crop Yield

| | | 0% | | 5% | | 10% | | 15% | |
|---|---|---|---|---|---|---|---|---|---|
| | | % | % | % | % | % | % | % | % |
| | | Increase | Decrease | Increase | Decrease | Increase | Decrease | Increase | Decrease |
| | | in NEV | in GHG | in NEV | in GHG | in NEV | in GHG | in NEV | in GHG |
| % | 0% | 0% | 0% | 4% | 4% | 8% | 7% | 12% | 10% |
| Decrease | 10% | 1% | 2% | 6% | 6% | 9% | 9% | 13% | 12% |
| in | 20% | 2% | 4% | 7% | 8% | 11% | 11% | 14% | 14% |
| Nitrogen | 30% | 4% | 6% | 8% | 10% | 12% | 13% | 15% | 16% |
| Application | 40% | 5% | 9% | 9% | 12% | 13% | 15% | 16% | 18% |
| Rate | 50% | 6% | 11% | 10% | 14% | 14% | 17% | 17% | 20% |
| | 60% | 7% | 13% | 11% | 16% | 15% | 19% | 18% | 22% |
| | 70% | 8% | 15% | 12% | 18% | 16% | 21% | 19% | 23% |

| | | 20% | | 25% | | 30% | |
|---|---|---|---|---|---|---|---|
| | | % | % | % | % | % | % |
| | | Increase | Decrease | Increase | Decrease | Increase | Decrease |
| | | in NEV | in GHG | in NEV | in GHG | in NEV | in GHG |
| % | 0% | 15% | 13% | 18% | 16% | 21% | 19% |
| Decrease | 10% | 16% | 15% | 19% | 18% | 22% | 20% |
| in | 20% | 17% | 17% | 20% | 19% | 23% | 22% |
| Nitrogen | 30% | 18% | 19% | 21% | 21% | 24% | 23% |
| Application | 40% | 19% | 20% | 22% | 23% | 25% | 25% |
| Rate | 50% | 20% | 22% | 23% | 25% | 26% | 27% |
| | 60% | 21% | 24% | 24% | 26% | 27% | 28% |
| | 70% | 22% | 26% | 25% | 28% | 28% | 30% |

TABLE 3

| | Amended | |
|---|---|---|
| | Values | Source of Amended Values |
| Agricultural Phase | | |
| N Application rate (kg/ha) | 25.78 | USDA 2000-2002 Average for soybeans |
| P2O5 application (kg/ha) | 54.55 | USDA 2000-2002 Average for soybeans |
| K2O application (kg/ha) | 93.03 | USDA 2000-2002 Average for soybeans |
| Lime application (kg/ha) | 448.34 | Hill et al. |
| Herbicide application rate (kg/ha) | 1.26 | Average USDA ARMS data 2000 and 2002 for soybeans |
| Insecticide (kg/ha) | 357.96 | Average USDA ARMS data 2000 and 2002 for soybeans |
| Seed rate (kg/ha) | 75.49 | Average USDA ARMS data 2000 and 2002 for soybeans |
| Biorefinery phase | | |
| Transportation of feedstock to biorefinery | 1.17 | Hill et al. |
| (MJ/L) | | |
| Coal (MJ/L) | 8.08 | Hill et al. |
| Capital (plant and equipment) (MJ/L) | 0.06 | Hill et al. |
| Process water (MJ/L) | 0.05 | 0.38 (ethanol value) * [[3 gal of diesel/gal water]/[4 gal of ethanol/gal water] * |
| | | [(Reported HV of ethanol) (MJ/L))/(Reported HV of biodiesel (MJ/L))] [1 gal/3.785 L]] = |
| | | 0.38 *[[3/4] * [21.20/32.90] * [1/3.79]] = 0.38 * [0.75 *0.64 * 0.264] = 0.048 |
| Effluent restoration (BOD energy cost to | 0.04 | 0.29 (ethanol value) * [[3 gal of diesel/gal water]/[4 gal of ethanol/gal water] * |
| PWTPs) (MJ/L) | | [(Reported HV of ethanol) (MJ/L))/(Reported HV of biodiesel (MJ/L))] [1 gal/3.785 L]] = |
| | | 0.29 *[[3/4] * [21.20/32.90] * [1/3.79]] = 0.048 |
| Totals | | |
| Crop yield (kg/ha) | 2680.15 | Average USDA data 2004-2006 for soybeans |
| Volumetric fuel yield (L/ha) | 544.00 | Hill et al. |
| Reported HV of biodiesel (MJ/L) | 32.90 | Hill et al. |
| Coproduct credits (MJ/L) | 21.90 | Hill et al. |

The above examples are provided to illustrate the invention but not to limit its scope. Other variants of the invention will be readily apparent to one of ordinary skill in the art and are encompassed by the appended claims and all their equivalents. All publications, patents, and patent applications cited herein are hereby incorporated by reference.

We claim:

1. A biofuel comprising ethanol wherein said ethanol has a net energy value (NEV), wherein the NEV of said ethanol is calculated using the formula NEV (MJ/LFuel)=[Biofuel Energy (MJ/Lfuel)+Coproduct Energy (MJ/Lfuel)]−[[[ΣiεFarmInputs(Embodied Energyi (MJ/kg)×Application Ratei (kg/ha))+ΣiεFarmInputs(Transport Energyi (MJ/kg)×Application Ratei (kg/ha))+[Nitrogen Embodied Energy (MJ/kgN)×Nitrogen Application Rate (kgN/ha)]+[Nitrogen Transport Energy (MJ/kgN)×Nitrogen Application Rate (kgN/ha)]+Farm Direct Energy (MJ/ha)+Farm Labor Energy (MJ/ha)+Farm Labor Transport Energy (MJ/ha)+Farm Machinery Energy (MJ/ha)+Inputs Packaging Energy (MJ/ha)]/[Crop Yield (kg/ha)×Production Yield (Lfuel/kg)]]+Biorefinery Energy (MJinput/Lfuel)], wherein said FarmInputs comprise one or more fertilizers, one or more herbicides, water, one or more insecticides, and seeds, and wherein said ethanol is derived from a genetically modified crop wherein said genetically modified crop is grown with a Crop Yield per Nitrogen Application Rate that is at least 8% higher than the Crop Yield per Nitrogen Application Rate of a non-genetically modified control crop, and wherein said ethanol derived from said genetically modified crop exhibits at least a 11% increase in NEV compared to the NEV of ethanol produced from said non-genetically modified control crop, and wherein said increase in NEV of said ethanol produced from said genetically modified crop is a result of a change in the Nitrogen Application Rate of the genetically modified crop or a change in the Crop Yield of the genetically modified crop or a combination thereof and said Biofuel Energy, Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and said Biorefinery Energy of the NEVs of the ethanol from said genetically modified crop and said non-genetically modified control crop are substantially the same.

2. The biofuel of claim 1, wherein the genetically modified crop is transgenic.

3. The biofuel of claim 1, wherein the genetically modified crop is selected from the group consisting of wheat, Miscanthus, corn, rice, barley, sorghum, millets, oats, rye, and sugarcane, rapeseed, castor bean, sunflower, safflower, soybean, palm, sugar beets, and cotton.

4. The biofuel of claim 2 wherein the transgenic crop includes recombinant DNA wherein the recombinant DNA encodes one or more proteins involved in nitrogen metabolism.

5. The biofuel of claim 4 wherein the one or more proteins are involved in nitrogen uptake or nitrogen assimilation.

6. The biofuel of claim 4 wherein the one or more proteins comprises alanine aminotransferase.

7. A biofuel comprising ethanol wherein said ethanol has a net greenhouse gas emission, wherein the net greenhouse gas emission of said ethanol is calculated using the formula net greenhouse gas emissions (kgCO2e/MJ)=[[[ΣᵢεFarmInputs(Input Emissionsᵢ (kgCO2e/kg)×Application Rateᵢ (kg/ha))+ΣᵢεFarmInputs(Transport Emissionsᵢ (kgCO2e/kg)×Application Rateᵢ (kg/ha))+[Fertilizer production emissions factor (kgCO2e/kgN)×Nitrogen application rate (kgN/ha)]+[Nitrification/denitrification emissions factor (kgCO2e/kgN)×Nitrogen Application Rate (kgN/ha)]+[Nitrogen Transport Emissions (kgCO2e/kgN)×Nitrogen Application Rate (kgN/ha)]+Farm Direct Emissions (kgCO2e/ha)+Farm Labor Transport Emissions (kgCO2e/ha)+Farm Machinery Emissions (kgCO2e/ha)+]/[Crop Yield (kg/ha)×Production Yield (L_{fuel}/kg)]]+Biorefinery Energy (kgCO2e/Lfuel)−Coproduct emissions (kgCO2e/L)]/HV of ethanol (MJ/L), wherein said FarmInputs comprise one or more fertilizers, one or more herbicides, water, one or more insecticides, and seeds, and wherein said ethanol is derived from a genetically modified crop wherein said genetically modified crop is grown with a Crop Yield per Nitrogen Application Rate that is at least 8% higher than the Crop Yield per Nitrogen Application Rate of a non-genetically modified control crop, and wherein said ethanol derived from said genetically modified crop exhibits at least a 6% decrease in the net greenhouse gas emissions compared to the net greenhouse gas emissions of ethanol produced from said non-genetically modified control crop, and wherein said decrease in net greenhouse gas emissions of said ethanol produced from said genetically modified crop is a result of a change in the Nitrogen Application Rate of the genetically modified crop or a change in the Crop Yield of the genetically modified crop or a combination thereof and said Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and said Biorefinery Energy of said net greenhouse gas emissions of the ethanol from the genetically modified crop and the ethanol from the non-genetically modified control crop are substantially the same.

8. The biofuel of claim 7, wherein the genetically modified crop is transgenic.

9. The biofuel of claim 7, wherein the genetically modified crop is selected from the group consisting of wheat, Miscanthus, corn, rice, barley, sorghum, millets, oats, rye, and sugarcane, rapeseed, castor bean, sunflower, safflower, soybean, palm, sugar beets, and cotton.

10. The biofuel of claim 8 wherein the transgenic crop includes recombinant DNA wherein the recombinant DNA encodes one or more proteins involved in nitrogen metabolism.

11. The biofuel of claim 10 wherein the one or more proteins are involved in nitrogen uptake or nitrogen assimilation.

12. The biofuel of claim 10 wherein the one or more proteins comprises alanine aminotransferase.

13. A biofuel comprising biodiesel wherein said biodiesel has a net energy value (NEV), wherein the NEV of said biodiesel is calculated using the formula NEV (MJ/LFuel)=[Biofuel Energy (MJ/Lfuel)+Coproduct Energy (MJ/Lfuel)]−[[[ΣiεFarmInputs(Embodied Energyi (MJ/kg)×Application Ratei (kg/ha))+ΣiεFarmInputs(Transport Energyi (MJ/kg)×Application Ratei (kg/ha))+[Nitrogen Embodied Energy (MJ/kgN)×Nitrogen Application Rate (kgN/ha)]+[Nitrogen Transport Energy (MJ/kgN)×Nitrogen Application Rate (kgN/ha)]+Farm Direct Energy (MJ/ha)+Farm Labor Energy (MJ/ha)+Farm Labor Transport Energy (MJ/ha)+Farm Machinery Energy (MJ/ha)+Inputs Packaging Energy (MJ/ha)]/[Crop Yield (kg/ha)×Production Yield (Lfuel/kg)]]+Biorefinery Energy (MJinput/Lfuel)], wherein said FarmInputs comprise one or more fertilizers, one or more herbicides, water, one or more insecticides, and seeds, and wherein said biodiesel is derived from a genetically modified crop wherein said genetically modified crop is grown with a Crop Yield per Nitrogen Application Rate that is at least 8% higher than the Crop Yield per Nitrogen Application Rate of a non-genetically modified control crop, and wherein said biodiesel derived from said genetically modified crop exhibits at least a 10% increase in NEV compared to the NEV of biodiesel produced from said non-genetically modified control crop, and wherein said increase in NEV of said biodiesel produced from said genetically modified crop is a result of a change in the Nitrogen Application Rate of the genetically modified crop or a change in the Crop Yield of the genetically modified crop or a combination thereof and said Biofuel Energy, Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and said Biorefinery Energy of the NEVs of said biodiesel from the genetically modified crop and from the non-genetically modified control crop are substantially the same.

14. The biofuel of claim 13, wherein the genetically modified crop is transgenic.

15. The biofuel of claim 13, wherein the genetically modified crop is selected from the group consisting of wheat, Miscanthus, corn, rice, barley, sorghum, millets, oats, rye, and sugarcane, rapeseed, castor bean, sunflower, safflower, soybean, palm, sugar beets, and cotton.

16. The biofuel of claim 14 wherein the transgenic crop includes recombinant DNA wherein the recombinant DNA encodes one or more proteins involved in nitrogen metabolism.

17. The biofuel of claim 16 wherein the one or more proteins are involved in nitrogen uptake or nitrogen assimilation.

18. The biofuel of claim 16 wherein the one or more proteins comprises alanine aminotransferase.

19. A biofuel comprising biodiesel wherein said ethanol has a net greenhouse gas emission, wherein the net greenhouse gas emission of said biodiesel is calculated using the formula net greenhouse gas emissions (kgCO2e/MJ)=[[[ΣᵢεFarmInputs(Input Emissionsᵢ (kgCO2e/kg)×Application Rateᵢ (kg/ha))+ΣᵢεFarmInputs(Transport Emissionsᵢ (kgCO2e/kg)×Application Rateᵢ (kg/ha))+[Fertilizer production emissions factor (kgCO2e/kgN)×Nitrogen application rate (kgN/ha)]+[Nitrification/denitrification emissions factor (kgCO2e/kgN)×Nitrogen Application Rate (kgN/ha)]+[Nitrogen Transport Emissions (kgCO2e/kgN)×Nitrogen Application Rate (kgN/ha)]+Farm Direct Emissions (kgCO2e/ha)+Farm Labor Transport Emissions (kgCO2e/ha)+Farm Machinery Emissions (kgCO2e/ha)+]/[Crop Yield (kg/ha)×Production Yield (L_{fuel}/kg)]]+Biorefinery Energy (kgCO2e/Lfuel)−Coproduct emissions (kgCO2e/L)]/HV of ethanol (MJ/L), wherein said FarmInputs comprise one or more fertilizers, one or more herbicides, water, one or more insecticides, and seeds, and wherein said biodiesel is derived from a genetically modified crop wherein said genetically modified crop is grown with a Crop Yield per Nitrogen Application Rate that is at least 8% higher than the Crop Yield per Nitrogen Application Rate of a non-genetically modified control crop, and wherein said biodiesel derived from said genetically modified crop exhibits at least a 10% decrease in the net greenhouse gas emissions compared to the net greenhouse gas emissions of biodiesel produced from said non-genetically modified control crop, and wherein said decrease in net greenhouse gas emissions of said biodiesel produced from said genetically modified crop is a result of a change in the Nitrogen Application Rate of the genetically modified crop or a change in the Crop Yield of the genetically modified crop or a combination thereof and said Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and said Biorefinery Energy of said net greenhouse gas emissions of the biodiesel from the genetically modified crop and the non-genetically modified control crop are substantially the same.

20. The biofuel of claim 19, wherein the genetically modified crop is transgenic.

21. The biofuel of claim 19, wherein the genetically modified crop is selected from the group consisting of wheat, Miscanthus, corn, rice, barley, sorghum, millets, oats, rye, and sugarcane, rapeseed, castor bean, sunflower, safflower, soybean, palm, sugar beets, and cotton.

22. The biofuel of claim 20 wherein the transgenic crop includes recombinant DNA wherein the recombinant DNA encodes one or more proteins involved in nitrogen metabolism.

23. The biofuel of claim 22 wherein the one or more proteins are involved in nitrogen uptake or nitrogen assimilation.

24. The biofuel of claim 22 wherein the one or more proteins comprises alanine aminotransferase.

25. A method of producing biofuel; comprising:
a) selecting genetically modified seed wherein said seed produces a genetically modified crop having a Crop Yield per Nitrogen Application Rate wherein the Crop Yield per Nitrogen Application Rate of said genetically modified crop is at least 8% higher than the Crop Yield per Nitrogen Application Rate of a non-genetically modified control crop;
b) planting said genetically modified seed to produce the genetically modified crop;
c) cultivating said genetically modified crop under conditions which maximize the Crop Yield per Nitrogen Application Rate of said genetically modified crop to produce a biofuel source;
d) harvesting said biofuel source;
e) preparing said biofuel source for processing to ethanol and
f) processing said biofuel source to produce ethanol wherein said ethanol has a net energy value (NEV), wherein said ethanol derived from said genetically modified crop exhibits at least a 11% increase in NEV compared to the NEV of ethanol produced from said non-genetically modified control crop, and wherein said increase in NEV of said ethanol produced from said genetically modified crop is a result of a change in the Nitrogen Application Rate of the genetically modified crop or a change in the Crop Yield of the genetically modified crop or a combination thereof and wherein the net energy value is calculated with NEV (MJ/LFuel)=[Biofuel Energy (MJ/Lfuel)+Coproduct Energy (MJ/Lfuel)]−[[[ΣiεFarmInputs(Embodied Energyi (MJ/kg)×Application Ratei (kg/ha))+ΣiεFarmInputs(Transport Energyi (MJ/kg)×Application Ratei (kg/ha))+[Nitrogen Embodied Energy (MJ/kgN)×Nitrogen Application Rate (kgN/ha)]+[Nitrogen Transport Energy (MJ/kgN)×Nitrogen Application Rate (kgN/ha)]+Farm Direct Energy (MJ/ha)+Farm Labor Energy (MJ/ha)+Farm Labor Transport Energy (MJ/ha)+Farm Machinery Energy (MJ/ha)+Inputs Packaging Energy (MJ/ha)]/[Crop Yield (kg/ha)×Production Yield (Lfuel/kg)]]+Biorefinery Energy (MJinput/Lfuel)], wherein said FarmInputs comprise one or more fertilizers, one or more herbicides, water, one or more insecticides, and seeds, and wherein said Biofuel Energy, Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and said Biorefinery Energy of the NEVs of the ethanol from the genetically modified crop and the non-genetically modified control crop are substantially the same.

26. The method of claim 25, wherein the genetically modified crop is transgenic.

27. The method of claim 25, wherein the genetically modified crop is selected from the group consisting of wheat, Miscanthus, corn, rice, barley, sorghum, millets, oats, rye, and sugarcane, rapeseed, castor bean, sunflower, safflower, soybean, palm, sugar beets, and cotton.

28. The method of claim 26, wherein the transgenic crop includes recombinant DNA wherein the recombinant DNA encodes one or more proteins involved in nitrogen metabolism.

29. The method of claim 28, wherein the one or more proteins are involved in nitrogen uptake or nitrogen assimilation.

30. The method of claim 28, wherein the one or more proteins comprises alanine aminotransferase.

31. A method of producing biofuel comprising:
a) selecting a genetically modified seed wherein said genetically modified seed produces a genetically modified crop having a Crop yield per Nitrogen Application Rate wherein said genetically modified crop is grown with a Crop Yield per Nitrogen Application Rate that is at least 8% higher than the Crop Yield per Nitrogen Application Rate of a non-genetically modified control crop;
b) planting said genetically modified seed to produce the genetically modified crop;
c) cultivating said genetically modified crop under conditions which maximize the Crop Yield per Nitrogen Application Rate of said genetically modified crop to produce a biofuel source;
d) harvesting said biofuel source;
e) preparing said biofuel source for processing to ethanol and
f) processing said biofuel source to produce ethanol wherein said ethanol has greenhouse gas emissions, wherein said ethanol derived from said genetically modified crop exhibits at least a 6% decrease in the net greenhouse gas emissions compared to the net greenhouse gas emissions of ethanol produced from said non-genetically modified control crop, and wherein said decrease in net greenhouse gas emissions of said ethanol produced from said genetically modified crop is a result of a change in the Nitrogen Application Rate of the genetically modified crop or a change in the Crop Yield of the genetically modified crop or a combination thereof, wherein the net greenhouse gas emissions of said ethanol is calculated using the formula: net greenhouse gas emissions (kgCO2e/MJ)=[[[ΣᵢεEFarmInputs(Input Emissionsᵢ (kgCO2e/kg)×Application Rateᵢ (kg/ha))+ΣᵢεFarmInputs(Transport Emissionsᵢ (kgCO2e/kg)×Application Rateᵢ (kg/ha))+[Fertilizer production emissions factor (kgCO2e/kgN)×Nitrogen application rate (kgN/ha)]+[Nitrification/denitrification emissions factor (kgCO2e/kgN)×Nitrogen Application Rate (kgN/ha)]+[Nitrogen Transport Emissions (kgCO2e/kgN)×Nitrogen Application Rate (kgN/ha)]+Farm Direct Emissions (kgCO2e/ha)++Farm Labor Transport Emissions (kgCO2e/ha)+Farm Machinery Emissions (kgCO2e/ha)+]/[Crop Yield (kg/ha)×Production Yield (L_{fue}/kg)]]+Biorefinery Energy (kgCO2e/Lfuel)−Coproduct emissions (kgCO2e/L)]/HV of ethanol (MJ/L), wherein said FarmInputs comprise one or more fertilizers, one or more herbicides, water, one or more insecticides, and seeds, and wherein said Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and said Biorefinery Energy of the net greenhouse gas emissions of the ethanol from genetically modified crop and the non-genetically modified control crop are substantially the same.

32. The method of claim 31, wherein the genetically modified crop is transgenic.

33. The method of claim 31, wherein the genetically modified crop is selected from the group consisting of wheat, Miscanthus, corn, rice, barley, sorghum, millets, oats, rye, and sugarcane, rapeseed, castor bean, sunflower, safflower, soybean, palm, sugar beets, and cotton.

34. The method of claim 32, wherein the transgenic crop includes recombinant DNA wherein the recombinant DNA encodes one or more proteins involved in nitrogen metabolism.

35. The method of claim 34, wherein the one or more proteins are involved in nitrogen uptake or nitrogen assimilation.

36. The method of claim 34, wherein the one or more proteins comprises alanine aminotransferase.

37. A method of producing biofuel; comprising:
a) selecting genetically modified seed wherein said seed produces a genetically modified crop having a Crop Yield per Nitrogen Application Rate wherein the Crop Yield per Nitrogen Application Rate of said genetically modified crop is at least 8% higher than the Crop Yield per Nitrogen Application Rate of a non-genetically modified control crop;
b) planting said genetically modified seed to produce the genetically modified crop;
c) cultivating said genetically modified crop under conditions which maximize the Crop Yield per Nitrogen Application Rate of said genetically modified crop to produce a biofuel source;
d) harvesting said biofuel source;
e) preparing said biofuel source for processing to biodiesel and
f) processing said biofuel source to produce biodiesel wherein said biodiesel has a net energy value (NEV), wherein said biodiesel derived from said genetically modified crop exhibits at least a 10% increase in NEV compared to the NEV of biodiesel produced from said non-genetically modified control crop, and wherein said increase in NEV of said biodiesel produced from said genetically modified crop is a result of a change in the Nitrogen Application Rate of the genetically modified crop or a change in the Crop Yield of the genetically modified crop or a combination thereof, wherein the net energy value is calculated with NEV (MJ/LFuel)=[Biofuel Energy (MJ/Lfuel)+Coproduct Energy (MJ/Lfuel)]−[[[ΣiεFarmInputs(Embodied Energyi (MJ/kg)×Application Ratei (kg/ha))+ΣiεFarmInputs(Transport Energyi (MJ/kg)×Application Ratei (kg/ha))+[Nitrogen Embodied Energy (MJ/kgN)×Nitrogen Application Rate (kgN/ha)]+[Nitrogen Transport Energy (MJ/kgN)×Nitrogen Application Rate (kgN/ha)]+Farm Direct Energy (MJ/ha)+Farm Labor Energy (MJ/ha)+Farm Labor Transport Energy (MJ/ha)+Farm Machinery Energy (MJ/ha)+Inputs Packaging Energy (MJ/ha)]/[Crop Yield (kg/ha)×Production Yield (Lfuel/kg)]]+Biorefinery Energy (MJinput/Lfuel)], wherein said FarmInputs comprise one or more fertilizers, one or more herbicides, water, one or more insecticides, and seeds, and wherein said Biofuel Energy, Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and said Biorefinery Energy of the NEVs of the biodiesel from genetically modified crop and said non-genetically modified control crop are substantially the same.

38. The method of claim 37, wherein the genetically modified crop is transgenic.

39. The method of claim 37, wherein the genetically modified crop is selected from the group consisting of wheat, Miscanthus, corn, rice, barley, sorghum, millets, oats, rye, and sugarcane, rapeseed, castor bean, sunflower, safflower, soybean, palm, sugar beets, and cotton.

40. The method of claim 38, wherein the transgenic crop includes recombinant DNA wherein the recombinant DNA encodes one or more proteins involved in nitrogen metabolism.

41. The method of claim 40, wherein the one or more proteins are involved in nitrogen uptake or nitrogen assimilation.

42. The method of claim 40, wherein the one or more proteins comprises alanine aminotransferase.

43. A method of producing biofuel comprising:
a) selecting a genetically modified seed wherein said genetically modified seed produces a genetically modified crop having a Crop yield per Nitrogen Application Rate wherein said genetically modified crop is grown with a Crop Yield per Nitrogen Application Rate that is at least 8% higher than the Crop Yield per Nitrogen Application Rate of a non-genetically modified control crop,;
b) planting said genetically modified seed to produce the genetically modified crop;
c) cultivating said genetically modified crop under conditions which maximize the Crop Yield per Nitrogen Application Rate of said genetically modified crop to produce a biofuel source;
d) harvesting said biofuel source;
e) preparing said biofuel source for processing to biodiesel and
f) processing said biofuel source to produce biodiesel wherein said biodiesel has greenhouse gas emissions, wherein said biodiesel derived from said genetically modified crop exhibits at least a 10% decrease in the net greenhouse gas emissions compared to the net greenhouse gas emissions of biodiesel produced from said non-genetically modified control crop, and wherein said decrease in net greenhouse gas emissions of said biodiesel produced from said genetically modified crop is a result of a change in the Nitrogen Application Rate of the genetically modified crop or a change in the Crop Yield of the genetically modified crop or a combination thereof, wherein the net greenhouse gas emissions is calculated with the formula: net greenhouse gas emissions (kgCO2e/MJ)=[[[ΣᵢεFarmInputs(Input Emissionsᵢ (kgCO2e/kg)×Application Rateᵢ(kg/ha))+ΣᵢεFarmInputs(Transport Emissions, (kgCO2e/kg)×Application Rateᵢ (kg/ha))+[Fertilizer production emissions factor (kgCO2e/kgN)×Nitrogen application rate (kgN/ha)]+[Nitrification/denitrification emissions factor (kgCO2e/kgN)×Nitrogen Application Rate (kgN/ha)]+[Nitrogen Transport Emissions (kgCO2e/kgN)×Nitrogen Application Rate (kgN/ha)]+Farm Direct Emissions (kgCO2e/ha)++Farm Labor Transport Emissions (kgCO2e/ha)+Farm Machinery Emissions (kgCO2e/ha)+]/[Crop Yield (kg/ha)×Production Yield (L_{fuel}/kg)]]+Biorefinery Energy (kgCO2e/Lfuel)−Coproduct emissions (kgCO2e/L)]/HV of ethanol (MJ/L), wherein said FarmInputs comprise one or more fertilizers, one or more herbicides, water, one or more insecticides, and seeds, and wherein said Coproduct Energy, Farm Inputs, Embodied Energy, Transport Energy, Nitrogen Embodied Energy, Nitrogen Transport Energy, Farm Direct Energy, Farm Labor Energy, Farm Labor Transport Energy, Farm Machinery Energy, Inputs Packaging Energy, Farm Inputs Embodied energy, Farm Inputs application rate and said Biorefinery Energy of the net greenhouse gas emissions of the biodiesel from genetically modified crop and the non-genetically modified control crop are substantially the same.

44. The method of claim 43, wherein the genetically modified crop is transgenic.

45. The method of claim 43, wherein the genetically modified crop is selected from the group consisting of wheat, Miscanthus, corn, rice, barley, sorghum, millets, oats, rye, and sugarcane, rapeseed, castor bean, sunflower, safflower, soybean, palm, sugar beets, and cotton.

46. The method of claim 44, wherein the transgenic crop includes recombinant DNA wherein the recombinant DNA encodes one or more proteins involved in nitrogen metabolism.

47. The method of claim 46, wherein the one or more proteins are involved in nitrogen uptake or nitrogen assimilation.

48. The method of claim 46, wherein the one or more proteins comprises alanine aminotransferase.
